(19) United States
(12) Patent Application Publication
(10) Pub. No.: US 2017/0342026 A1
(43) Pub. Date: Nov. 30, 2017

(54) ISOPROPOXY-PHENYLGUANIDINE SERIES SALT AND APPLICATION THEREOF IN PREPARING GROWTH PROMOTERS FOR ANIMAL FEED

(71) Applicant: GUANGZHOU INSIGHTER BIOTECHNOLOGY CO., LTD., Guangzhou, Guangdong (CN)

(72) Inventors: Xianfeng Peng, Guangzhou (CN); Zonghua QIN, Guangzhou (CN)

(73) Assignee: GUANGZHOU INSIGHTER BIOTECHNOLOGY CO., LTD., Guangzhou, Guangdong (CN)

(21) Appl. No.: 15/535,711

(22) PCT Filed: Mar. 18, 2015
(86) PCT No.: PCT/CN2015/074453

§ 371 Date: Jun. 14, 2017

(30) Foreign Application Priority Data
Feb. 16, 2015 (CN) 201510084594.3

(51) Int. Cl. C07C 281/18; A23K 50/75; A23K 50/70; A23K 20/10; A23K 50/30

(52) U.S. Cl. CPC C07C 281/18; A23K 20/10; A23K 50/30; A23K 50/70; A23K 50/75

(57) ABSTRACT

Isopropoxy-phenylguanidine series salt as shown by the Formula (I) and an application thereof in preparing growth promoters for animal feed, wherein R is DL-lactic acid, methanesulfonic acid, 2-hydroxyethanesulfonic acid, citric acid, tartaric acid, benzoic acid, succinic acid, fumaric acid, maleic acid, acetic acid, sulfuric acid, phosphoric acid, or oxalic acid.

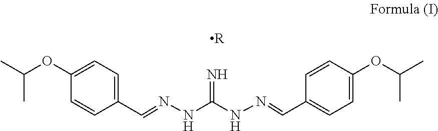

### CROSS-REFERENCE TO RELATED APPLICATIONS

This is a U.S. national stage application of the International Patent Application No. PCT/CN2015/074453, filed Mar. 18, 2015, which claims priority to Chinese Patent Application No. 201510084594.3, filed Feb. 16, 2015, both of which are incorporated herein by reference in their entireties.

### FIELD OF THE INVENTION

This invention relates to the field of feed for livestock and poultry, in particular, for example, the present invention relates to a series of salts of isopropoxy-phenylguanidine and uses thereof in the preparation of growth promoters for a feed for animal.

### BACKGROUND OF THE INVENTION

Growth promoters for feed, including bioactive substances with various action mechanisms and various chemical structures and formulations thereof, have significant contributions to the improvements of the feed conversion rate as well as the production efficiency of animal cultivation. However, many antibiotic growth promoters that are structurally relevant or cross-resistant to antibiotics used in human clinical medicine have been or are about to be banned due to the transfer of drug resistance.

Therefore, it is a problem that how to screen out a safe and efficient growth promoter for a feed for dedicated animal, which are not used in human medicine and not cross-resistant to antibiotics for use for human, for example, preferably a non-antibiotic and non-hormonal growth promoter for a feed. Fortunately, the present disclosure solved this problem.

### SUMMARY OF THE INVENTION

In one aspect, provided herein is a salt of isopropoxy-phenylguanidine having Formula (I):

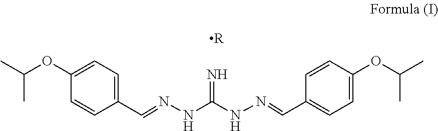

wherein R is DL-lactic acid, methanesulfonic acid, 2-hydroxyethanesulfonic acid, citric acid, tartaric acid, benzoic acid, succinic acid, fumaric acid, maleic acid, acetic acid, sulfuric acid, phosphoric acid, or oxalic acid.

In another aspect, provided herein is use of the salt of isopropoxy-phenylguanidine according to claim 1 in the preparation of a growth promoter for a feed for animal.

In certain embodiments, the animal is a farmed animal in any one of growth stages.

In some embodiments, the farmed animal is duck, pig, or chicken.

In certain embodiments, the concentration of the salt of isopropoxy-phenylguanidine in the feed for animal is from 0.1 ppm to 500 ppm.

### DETAILED DESCRIPTION OF THE INVENTION

In one aspect, provided herein is a series of salts of isopropoxy-phenylguanidine, which can efficiently promote the growth of animals.

In some embodiments, any one of the series of the salts of isopropoxy-phenylguanidine having Formula (I):

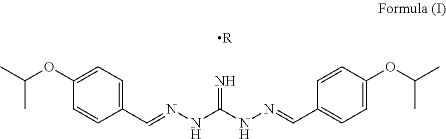

wherein R is DL-lactic acid, methanesulfonic acid, 2-hydroxyethanesulfonic acid, citric acid, tartaric acid, benzoic acid, succinic acid, fumaric acid, maleic acid, acetic acid, sulfuric acid, phosphoric acid, or oxalic acid.

In other aspect, provided herein is use of any one of the series of salts of isopropoxy-phenylguanidine having Formula (I) in the preparation of a growth promoter for a feed for animal.

In some embodiments, the animal is a farmed animal in any one of growth stages, and the farmed animal is duck, pig, chicken or any one of other farmed animals.

In some embodiments, the concentration of any one of the series of salts of isopropoxy-phenylguanidine in the feed for animal is from 0.1 ppm to 500 ppm, and the feed is a complete formula feed.

In some embodiments, the salt of isopropoxy-phenylguanidine of Formula (I) shows low toxicity or non-toxicity to animals, which makes it suitable to be used as a growth promoter in a feed and has a very good prospect for use in cultivation industry.

The present disclosure is first found that the production performance of ducks is greatly improved when they are fed with any one of the salts of isopropoxy-phenylguanidine through feeding tests. It is also disclosed herein that the production performance of pigs or chickens can be improved when they are fed with such salts of isopropoxy-phenylguanidine. Therefore, the series of salts of isopropoxy-phenylguanidine disclosed herein can be used in the preparation of growth promoters for feed and have broad prospects for use.

### Examples

For further descriptions of the present invention, the following examples and test examples are presented, but these examples and test examples should not be construed to limit the scope of the invention.

### Example 1. Preparation of free 2,2′-bis[[4-(isopropoxy)phenyl]methylene]-diaminoguanidine

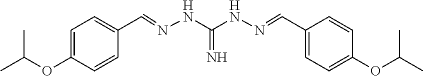 [SMILES: CC(C)Oc1ccc(/C=N/NC(=N)N/N=C/c2ccc(OC(C)C)cc2)cc1]

To a flask was added 2,2′-bis[[4-(isopropoxy)phenyl]methylene]-diaminoguanidine hydrochloride (100 g, water content of about 15%), ethanol (95%, 750 mL) and NaOH (12.5 g), then 2,2′-bis[[4-(isopropoxy)phenyl]methylene]-diaminoguanidine was gradually dissolved and NaCl was precipitated. The reaction mixture was stirred for 0.5 hours, filtered and the filtrate was added into water (3 L) dropwise to precipitate a solid. The resulted solid was collected by filtration and dried at 45° C. to afford the title compound 2,2′-bis[[4-(isopropoxy)phenyl]methylene]-diaminoguanidine (70 g).

### Preparations of R salts of 2,2′-bis[[4-(isopropoxy)phenyl]methylene]-diaminoguanidine of Formula (I)

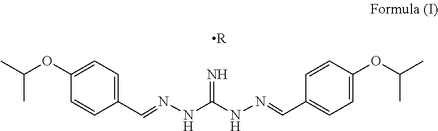

wherein R is DL-lactic acid, methanesulfonic acid, 2-hydroxyethanesulfonic acid, citric acid, tartaric acid, benzoic acid, succinic acid, fumaric acid, maleic acid, acetic acid, sulfuric acid, phosphoric acid, or oxalic acid.

### Example 2. Preparation of 2,2′-bis[[4-(1-methylethoxy)phenyl]methylene]-carbonimidic dihydrazide.2-hydroxypropanoic acid/2,2′-bis[[4-(isopropoxy)phenyl]methylene]-diaminoguanidine DL-lactate

To a stirred solution of free 2,2′-bis[[4-(isopropoxy)phenyl]methylene]-diaminoguanidine (1.6 g) and ethanol (95%, 8 mL) was added DL-lactic acid (0.4 g) at room temperature, then the reaction mixture was stirred for 2 h. The resulted solid was collected by filtration and washed with ethanol (95%) and water, and then dried at 45° C. overnight to afford the title compound 2,2′-bis[[4-(isopropoxy)phenyl]methylene]-diaminoguanidine DL-lactate, which is also named isopropoxy-phenylguanidine lactate (1.1 g, 55%). ¹H NMR (500 MHz, d₆-DMSO) δ7.99 (s, 2H), δ7.67 (d, 4H), δ6.92 (d, 4H), δ6.56 (br, 2H), δ4.63-4.67 (m, 2H), δ4.01 (q, 1H), δ1.28 (d, 12H), δ1.22 (d, 3H).

### Example 3. Preparation of 2,2′-bis[[4-(1-methylethoxy)phenyl]methylene]-carbonimidic dihydrazide.methanesulfonic acid/2,2′-bis[[4-(isopropoxy)phenyl]methylene]-diaminoguanidine methanesulfonate

To a stirred solution of free 2,2′-bis[[4-(isopropoxy)phenyl]methylene]-diaminoguanidine (1.6 g) and ethanol (95%, 8 mL) was added methanesulfonic acid (0.4 g) at room temperature, then the reaction mixture was stirred for 2 h. The resulted solid was collected by filtration and washed with ethanol (95%) and water, and then dried at 45° C. overnight to afford the title compound 2,2′-bis[[4-(isopropoxy)phenyl]methylene]-diaminoguanidine methanesulfonate, which is also named isopropoxy-phenylguanidine methanesulfonate (1.4 g, 70%). ¹H NMR (500 MHz, d₆-DMSO) δ11.8 (br, 1H), δ8.39 (s, 2H), δ8.26 (br, 2H), δ7.85 (d, 4H), δ7.01 (d, 4H), δ4.70-4.75 (m, 2H), δ2.41 (s, 3H), δ1.28 (d, 12H).

### Example 4. Preparation of 2,2′-bis[[4-(1-methylethoxy)phenyl]methylene]-carbonimidic dihydrazide.2-hydroxyethanesulfonic acid/2,2′-bis[[4-(isopropoxy)phenyl]methylene]-diaminoguanidine 2-hydroxyethylsulfonate

To a stirred solution of free 2,2′-bis[[4-(isopropoxy)phenyl]methylene]-diaminoguanidine (1.6 g) and ethanol (95%, 8 mL) was added hydroxyethanesulfonic acid (0.5 g) at room temperature. The reaction mixture was stirred for 2 hours. The resulted solid was collected by filtration and washed with ethanol (95%) and water, and then dried at 45° C. overnight to afford the title compound 2,2′-bis[[4-(isopropoxy)phenyl]methylene]-diaminoguanidine 2-hydroxyethylsulfonate, which is also named isopropoxy-phenylguanidine 2-hydroxyethylsulfonate (1.4 g, 66%). ¹H NMR (500 MHz, d₆-DMSO) δ11.58 (br, 1H), δ8.26 (s, 2H), δ8.11 (br, 2H), δ7.82 (d, 4H), δ7.01 (d, 4H), δ4.69-4.74 (m, 2H), δ3.64 (t, 2H), δ2.62 (t, 2H), δ1.29 (d, 12H).

### Example 5. Preparation of 2,2′-bis[[4-(1-methylethoxy)phenyl]methylene]-carbonimidic dihydrazide.2-hydroxypropane-1,2,3-tricarboxylic acid/2,2′-bis[[4-(isopropoxy)phenyl]-methylene]-diaminoguanidine citrate

To a stirred solution of free 2,2′-bis[[4-(isopropoxy)phenyl]methylene]-diaminoguanidine (1.6 g) and ethanol (95%, 8 mL) was added citric acid (0.8 g) at room temperature, then the reaction mixture was stirred for 2 hours. The solid was collected by filtration and washed with ethanol (95%) and water, and then dried at 45° C. overnight to afford the title compound 2,2′-bis[[4-(isopropoxy)phenyl]methylene]-diaminoguanidine citrate, which is also named isopropoxy-phenylguanidine citrate (1.8 g, 75%). ¹H NMR (500 MHz, d₆-DMSO) δ11.29 (br, 1H), δ8.09 (s, 2H), δ7.72 (d, 4H), δ7.11 (br, 2H), δ6.95 (d, 4H), δ4.64-4.71 (m, 2H), δ2.51-2.62 (m, 2H), δ1.29 (d, 12H).

### Example 6. Preparation of 2,2′-bis[[4-(1-methylethoxy)phenyl]methylene]-carbonimidic dihydrazide.(2R,3S)-2,3-dihydroxysuccinic acid/2,2′-bis[[4-(isopropoxy)phenyl]methylene]-diaminoguanidine tartrate

To a stirred solution of free 2,2′-bis[[4-(isopropoxy)phenyl]methylene]-diaminoguanidine (1.6 g) and ethanol (95%, 8 mL) was added tartaric acid (0.6 g) at room temperature. The reaction mixture was stirred for 2 hours. The solid was collected by filtration and washed with ethanol (95%) and water, and then dried at 45° C. overnight to afford the title compound 2,2′-bis[[4-(isopropoxy)phenyl]methylene]-diaminoguanidine tartrate, which is also named isopropoxy-phenylguanidine tartrate (1.5 g, 68%). ¹H NMR (500 MHz, d₆-DMSO) δ10.15 (br, 1H), δ8.08 (s, 2H), δ7.65 (d, 4H), δ7.47 (br, 2H), δ6.94 (d, 4H), δ4.65-4.70 (m, 2H), δ4.18 (s, 1H), δ1.28 (d, 12H).

### Example 7. Preparation of 2,2′-bis[[4-(1-methylethoxy)phenyl]methylene]-carbonimidic dihydrazide.benzoic acid/2,2′-bis[[4-(isopropoxy)phenyl]methylene]-diaminoguanidine benzoate

To a stirred solution of free 2,2′-bis[[4-(isopropoxy)phenyl]methylene]-diaminoguanidine (1.6 g) and ethanol (95%, 8 mL) was added benzoic acid (0.5 g) at room temperature, then the reaction mixture was stirred for 2 hours. The resulted solid was collected by filtration and washed with ethanol (95%) and water, and then dried at 45° C. overnight to afford the title compound 2,2′-bis[[4-(isopropoxy)phenyl]methylene]-diaminoguanidine benzoate, which is also named isopropoxy-phenylguanidine benzoate (0.5 g, 23%). ¹H NMR (500 MHz, d₆-DMSO) δ12.11 (br, 1H), δ8.00 (s, 2H), δ7.95 (t, 2H), δ7.67 (d, 4H), δ7.60 (t, 1H), δ7.50 (t, 2H), δ6.92 (d, 4H), δ6.59 (s, 2H), δ4.63-4.68 (m, 2H), δ1.28 (d, 12H).

### Example 8. Preparation of 2,2′-bis[[4-(1-methylethoxy)phenyl]methylene]-carbonimidic dihydrazide.succinic acid/2,2′-bis[[4-(isopropoxy)phenyl]methylene]-diaminoguanidine succinate

To a stirred solution of free 2,2′-bis[[4-(isopropoxy)phenyl]methylene]-diaminoguanidine (1.6 g) and ethanol (95%, 8 mL) was added succinic acid (0.28 g) at room temperature, then the reaction mixture was stirred for 2 hours. The resulted solid was collected by filtration and washed with ethanol (95%) and water, and then dried at 45° C. overnight to afford the title compound 2,2′-bis[[4-(isopropoxy)phenyl]methylene]-diaminoguanidine succinate, which is also named isopropoxyphenyl guanidine succinate (1.6 g, 76%). ¹H NMR (500 MHz, d₆-DMSO) δ11.80 (br, 1H), δ8.00 (s, 2H), δ7.66 (d, 4H), δ6.92 (d, 4H), δ6.56 (br, 2H), δ4.63-4.68 (m, 2H), δ2.40 (s, 2H), δ1.28 (d, 12H).

### Example 9. Preparation of 2,2′-bis[[4-(1-methylethoxy)phenyl]methylene]-carbonimidic dihydrazide.fumaric acid/22′-bis[[4-(isopropoxy)phenyl]methylene]-diaminoguanidine fumarate

To stirred solution of free 2,2′-bis[[4-(isopropoxy)phenyl]methylene]-diaminoguanidine (1.6 g) and ethanol (95%, 8 mL) was added fumaric acid (0.24 g) at room temperature, then the reaction mixture was stirred overnight. The resulted solid was collected by filtration, washed with ethanol (95%) and dried at 45° C. overnight to afford the title compound 2,2′-bis[[4-(isopropoxy)phenyl]methylene]-diaminoguanidine fumarate, which is also named isopropoxy-phenylguanidine fumarate (1.78 g, 96.7%). ¹H NMR (500 MHz, d₆-DMSO) δ8.06 (s, 2H), δ7.72 (d, 4H), δ7.19 (br, 2H), δ6.94 (d, 4H), δ6.60 (s, 1H), δ4.63-4.71 (m, 2H), δ1.28 (d, 12H).

### Example 10. Preparation of 2,2′-bis[[4-(1-methylethoxy)phenyl]methylene]-carbonimidic dihydrazide.maleic acid/2,2′-bis[[4-(isopropoxy)phenyl]methylene]-diamninoguanidine maleate

To a stirred solution of free 2,2′-bis[[4-(isopropoxy)phenyl]methylene]-diaminoguanidine (1.6 g) and ethanol (95%, 8 mL) was added maleic acid (0.24 g) at room temperature, then the reaction mixture was stirred overnight. The resulted solid was collected by filtration, washed with ethanol (95%) and then dried at 45° C. overnight to afford the title compound 2,2′-bis[[4-(isopropoxy)phenyl]methylene]-diaminoguanidine maleate, which is also named isopropoxy-phenylguanidine maleate (1.64 g, 89.1%). ¹H NMR (500 MHz, d₆-DMSO) δ8.17 (s, 2H), δ7.77 (d, 4H), δ7.54 (br, 2H), δ6.97 (d, 4H), δ6.02 (s, 1H), δ4.66-4.73 (m, 2H), δ1.28 (d, 12H).

### Example 11. Preparation of 2,2′-bis[[4-(1-methylethoxy)phenyl]methylene]-carbonimidic dihydrazide. acetic acid/2,2′-bis[[4-(isopropoxy)phenyl]methylene]-diaminoguanidine acetate

To a stirred solution of acetic acid (0.25 g) in water (20 mL) was added free 2,2′-bis[[4-(isopropoxy)phenyl]methylene]-diaminoguanidine (1.6 g) at room temperature, then ethanol (95%, 20 mL) was added, the reaction mixture was stirred overnight. The resulted solid was collected by filtration, washed with water and then dried at 45° C. overnight to afford the title compound 2,2′-bis[[4-(isopropoxy)phenyl]methylene]-diaminoguanidine acetate, which is also named isopropoxy-phenylguanidine acetate (1.31 g, 70.8%). ¹H NMR (500 MHz, d₆-DMSO) δ7.98 (s, 2H), δ7.65 (d, 4H), δ6.91 (d, 4H), δ6.44 (br, 2H), δ4.63-4.67 (m, 2H), δ1.90 (s, 3H), δ1.28 (d, 12H).

### Example 12. Preparation of 2,2′-bis[[4-(1-methylethoxy)phenyl]methylene]-carbonimidic dihydrazide.sulfuric acid/2,2′-bis[[4-(isopropoxy)phenyl]methylene]-diaminoguanidine sulfate

To a stirred solution of free 2,2′-bis[[4-(isopropoxy)phenyl]methylene]-diaminoguanidine (1.6 g) and ethanol (95%, 8 mL) was added sulfuric acid (0.21 g) at room temperature. After complete dissolution of the starting materials, the reaction mixture was stirred for 0.5 hours, then water was added and a solid was precipitated. The resulted solid was collected by filtration and washed with ethanol (95%) and water, then dried at 45° C. overnight to afford the title compound 2,2′-bis[[4-(isopropoxy)phenyl]methylene]-diaminoguanidine sulfate, which is also named isopropoxy-phenylguanidine sulfate (1.6 g, 88.4%). ¹H NMR (500 MHz, d₆-DMSO) δ11.4 (br, 1H), δ8.13 (s, 2H), δ7.95 (d, 4H), δ7.55 (br, 2H), δ6.95 (d, 4H), δ4.65-4.72 (m, 2H), δ1.28 (d, 12H).

### Example 13. Preparation of 2,2′-bis[[4-(1-methylethoxy)phenyl]methylene]-carbonimidic dihydrazide.phosphoric acid/2,2′-bis[[4-(isopropoxy)phenyl]methylene]-diaminoguanidine phosphate

To a stirred solution of free 2,2′-bis[[4-(isopropoxy)phenyl]methylene]-diaminoguanidine (1.6 g) and ethanol (95%, 8 mL) was added phosphoric acid (0.14 g) at room temperature. After complete dissolution of the starting materials, the reaction mixture was stirred overnight. The resulted solid was collected by filtration, and washed with ethanol (95%) and water, then dried at 45° C. overnight to afford the title compound 2,2′-bis[[4-(isopropoxy)phenyl] methylene]-diaminoguanidine phosphate, which is also named isopropoxy-phenylguanidine phosphate (1.33 g, 76.4%). ¹H NMR (500 MHz, d₆-DMSO) δ8.03 (s, 2H), δ7.69 (d, 4H), δ6.92-6.94 (m, 6H), δ4.63-4.70 (m, 2H), δ1.28 (d, 12H).

### Example 14. Preparation of 2,2′-bis[[4-(1-methylethoxy)phenyl]methylene]-carbonimidic dihydrazide.oxalic acid/2,2′-bis[[4-(isopropoxy)phenyl]methylene]-diaminoguanidine oxalate

To a stirred solution of free 2,2′-bis[[4-(isopropoxy)phenyl]methylene]-diaminoguanidine (1.6 g) and ethanol (95%, 8 mL) was added oxalic acid (0.19 g) at room temperature. The reaction mixture was stirred overnight. The resulted solid was collected by filtration, washed with ethanol (95%) and dried at 45° C. overnight to afford the title compound 2,2′-bis[[4-(isopropoxy)phenyl] methylene]-diaminoguanidine oxalate, which is also named isopropoxy-phenylguanidine oxalate (1.67 g, 93.3%). ¹H NMR (500 MHz, d₆-DMSO) δ8.08 (s, 2H), δ7.73 (d, 4H), δ7.26 (br, 2H), δ6.94 (d, 4H), δ4.64-4.71 (m, 2H), δ1.28 (d, 12H).

### Example 15. Effects of the Series of Salts of Isopropoxy-Phenylguanidine on Production Performance of Broilers

One thousand and five hundred (1500) 1-day-old broilers were divided into 15 groups and each group with 100 broilers. Different types of salts of isopropoxy-phenylguanidine were added to the same basic diets. The broilers were free feeding and drinking and continued feeding for 30 days, then to collect statistics of the production performance and the survival rate of broilers in each group. The test design and results are shown in Table 1. It can be seen that the effects of different salts of isopropoxy-phenylguanidine on the production performance of broilers are different, wherein the improvement of the production performance of the group of lactate, methanesulfonate or 2-hydroxyethanesulfonate of isopropoxy-phenylguanidine is most significant, and the effect of any one of above salts is better than hydrochloride, while the effects of other salts are inferior to hydrochloride.

TABLE 1
Test design and effects of the series of salts of isopropoxy-phenylguanidine on
production performance of broilers.

| | | | | Average | Average | |
|---|---|---|---|---|---|---|
| | Number | | | daily | feed | |
| | of | | Concentration* | weight | conversion | Survival |
| Group | broilers | Compound | (ppm) | gain (g) | ratio | rate (%) |
| 1 | 100 | — | 0 | 41.3 | 2.147 | 100 |
| 2 | 100 | isopropoxy-phenylguanidine | 50 | 47.9 | 2.006 | 100 |
| | | lactate | | | | |
| 3 | 100 | isopropoxy-phenylguanidine | 50 | 48.8 | 1.999 | 100 |
| | | methanesulfonate | | | | |
| 4 | 100 | isopropoxy-phenylguanidine | 50 | 49.1 | 1.987 | 100 |
| | | 2-hydroxyethylsulfonate | | | | |
| 5 | 100 | isopropoxy-phenylguanidine | 50 | 44.5 | 2.125 | 100 |
| | | citrate | | | | |
| 6 | 100 | isopropoxy-phenylguanidine | 50 | 43.9 | 2.131 | 98 |
| | | tartrate | | | | |
| 7 | 100 | isopropoxy-phenylguanidine | 50 | 44.7 | 2.126 | 100 |
| | | benzoate | | | | |
| 8 | 100 | isopropoxy-phenylguanidine | 50 | 43.6 | 2.143 | 100 |
| | | succinate | | | | |
| 9 | 100 | isopropoxy-phenylguanidine | 50 | 44.1 | 2.136 | 99 |
| | | fumarate | | | | |
| 10 | 100 | isopropoxy-phenylguanidine | 50 | 44.1 | 2.137 | 100 |
| | | maleate | | | | |
| 11 | 100 | isopropoxy-phenylguanidine | 50 | 43.2 | 2.129 | 99 |
| | | acetate | | | | |
| 12 | 100 | isopropoxy-phenylguanidine | 50 | 43.7 | 2.131 | 100 |
| | | sulfate | | | | |
| 13 | 100 | isopropoxy-phenylguanidine | 50 | 44.0 | 2.139 | 100 |
| | | phosphate | | | | |
| 14 | 100 | isopropoxy-phenylguanidine | 50 | 43.1 | 2.145 | 100 |
| | | oxalate | | | | |
| 15 | 100 | isopropoxy-phenylguanidine | 50 | 46.6 | 2.087 | 100 |
| | | hydrochloride | | | | |

*Each test concentration was determined by the content of isopropoxy-phenylguanidine in the feed for broilers.

### Example 16. Effects of Different Concentrations of Isopropoxy-Phenylguanidine Salts on Production Performance of Broilers

Eight hundred (800) broilers aged 1 day were divided into 8 groups and each group with 100 broilers. Different concentrations of isopropoxy-phenylguanidine methanesulfonate were added to the same basic diets. The broilers were free feeding and drinking and continued feeding for 30 days, then to collect statistics of the production performance and the survival rate of broilers in each group. The test design and results of production performance are shown in Table 2. It can be seen that isopropoxy-phenylguanidine methanesulfonate in different concentrations can significantly improve the production performance of broilers, and it has a dose effect on the improvement of production performance, that is, the higher of the concentration, and the more significant of the effect. Further, methanesulfonate with high concentrations have no negative impacts on the production performance.

TABLE 2
Effects of different concentrations of isopropoxy-phenylguanidine
methanesulfonate on production performance of broilers.

| | | | | | Daily | |
|---|---|---|---|---|---|---|
| | Number | | | | weight | Feed |
| | of | | Concentration* | Survival | gain | conversion |
| Group | broilers | Compound | (ppm) | rate (%) | (g) | ratio |
| 1 | 100 | — | 0 | 100 | 41.2 | 2.161 |
| 2 | 100 | isopropoxy-phenylguanidine | 5 | 100 | 42.3 | 2.150 |
| | | methanesulfonate | | | | |
| 3 | 100 | isopropoxy-phenylguanidine | 10 | 100 | 43.4 | 2.138 |
| | | methanesulfonate | | | | |
| 4 | 100 | isopropoxy-phenylguanidine | 20 | 100 | 44.5 | 2.119 |
| | | methanesulfonate | | | | |
| 5 | 100 | isopropoxy-phenylguanidine | 50 | 100 | 48.9 | 2.087 |
| | | methanesulfonate | | | | |
| 6 | 100 | isopropoxy-phenylguanidine | 100 | 100 | 49.6 | 2.076 |
| | | methanesulfonate | | | | |
| 7 | 100 | isopropoxy-phenylguanidine | 200 | 100 | 49.2 | 2.071 |
| | | methanesulfonate | | | | |
| 8 | 100 | isopropoxy-phenylguanidine | 500 | 100 | 49.5 | 2.073 |
| | | methanesulfonate | | | | |

*Each test concentration was determined by the content of isopropoxy-phenylguanidine in the feed for broilers.

### Example 17. Effects of the Salts of Isopropoxy-Phenylguanidine in a Feed for Pigs

One hundred and twenty (120) Duroc×Landrace×Large White piglets aged 28 days with similar body weight were randomly divided into 6 groups, each with 20 pigs. Different types of salts of isopropoxy-phenylguanidine were added to the feeds respectively in accordance with Table 3. The pigs were free feeding and drinking during the test period of 28 days. The results show that different types of salts of isopropoxy-phenylguanidine can improve the weight gain of the pigs, and can also improve the feed conversion ratio in different degrees, wherein the effect of the salt of isopropoxy-phenylguanidine selected from lactate, methanesulfonate or 2-hydroxyethanesulfonate on the production performance is better than hydrochloride, followed by citrate. The test grouping and results are shown in Table 3.

TABLE 3
Grouping design and effects of different salts of isopropoxy-
phenylguanidine on production performance of pigs.

| | | | | Average | |
|---|---|---|---|---|---|
| | Number | | Concen- | daily | Feed |
| | of test | | tration* | weight | conversion |
| Group | pigs | Additive | (ppm) | gain (g) | ratio |
| 1 | 20 | — | — | 479 | 1.835 |
| 2 | 20 | isopropoxy- | 100 | 535 | 1.701 |
| | | phenylguanidine | | | |
| | | lactate | | | |
| 3 | 20 | isopropoxy- | 100 | 529 | 1.713 |
| | | phenylguanidine | | | |
| | | methanesulfonate | | | |
| 4 | 20 | isopropoxy- | 100 | 537 | 1.697 |
| | | phenylguanidine 2- | | | |
| | | hydroxyethylsulfonate | | | |
| 5 | 20 | isopropoxy- | 100 | 491 | 1.803 |
| | | phenylguanidine | | | |
| | | citrate | | | |
| 6 | 20 | isopropoxy- | 100 | 512 | 1.754 |
| | | phenylguanidine | | | |
| | | hydrochloride | | | |

*Each concentration was determined by the content of isopropoxy-phenylguanidine in the feed for pigs.

### Example 18. Effects of the Salts of Isopropoxy-Phenylguanidine in a Feed for Ducks

Six hundred (600) Cherry Valley ducks aged 1 day were randomly divided into 6 groups, each with 100 ducks. Different types of salts of isopropoxy-phenylguanidine were added to the feed according to Table 4. The ducks were free feeding and drinking during the test period of 28 days. The results show that different types of salts of isopropoxy-phenylguanidine can improve the weight gain of the test ducks, and can also improve the feed conversion ratio in different degrees, wherein the effect of the salt of isopropoxy-phenylguanidine selected from lactate, methanesulfonate or 2-hydroxyethanesulfonate on the production performance is the best and also better than hydrochloride, followed by phosphate. The test grouping and results are shown in Table 4.

TABLE 4
Grouping design and effects of different salts of isopropoxy-
phenylguanidine on production performance of meat-type ducks.

| | | | | Average | |
|---|---|---|---|---|---|
| | Number | | Concen- | daily | Feed |
| | of test | | tration * | weight | conversion |
| Group | ducks | Additive | (ppm) | gain (g) | ratio |
| 1 | 100 | — | — | 57.9 | 1.999 |
| 2 | 100 | isopropoxy- | 50 | 67.8 | 1.827 |
| | | phenylguanidine | | | |
| | | lactate | | | |
| 3 | 100 | isopropoxy- | 50 | 68.4 | 1.831 |
| | | phenylguanidine | | | |
| | | methanesulfonate | | | |
| 4 | 100 | isopropoxy- | 50 | 67.9 | 1.835 |
| | | phenylguanidine 2- | | | |
| | | hydroxyethylsulfonate | | | |
| 5 | 100 | isopropoxy- | 50 | 61.2 | 1.926 |
| | | phenylguanidine | | | |
| | | phosphate | | | |
| 6 | 100 | isopropoxy- | 50 | 65.3 | 1.874 |
| | | phenylguanidine | | | |
| | | hydrochloride | | | |

* Each concentration was determined by the content of isopropoxy-phenylguanidine in the feed for ducks.

What is claimed is:

1. A salt of isopropoxy-phenylguanidine having Formula (I): 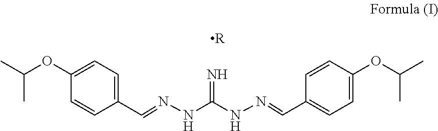
wherein R is DL-lactic acid, methanesulfonic acid, 2-hydroxyethanesulfonic acid, citric acid, tartaric acid, benzoic acid, succinic acid, fumaric acid, maleic acid, acetic acid, sulfuric acid, phosphoric acid, or oxalic acid.

2. Use of the salt of isopropoxy-phenylguanidine according to claim 1 in the preparation of a growth promoter for a feed for animal.

3. The use according to claim 2, wherein the animal is a farmed animal in any one of growth stages.

4. The use according to claim 3, wherein the farmed animal is duck, pig, or chicken.

5. The use according to claim 2, wherein the concentration of the salt of isopropoxy-phenylguanidine in the feed for animal is from 0.1 ppm to 500 ppm.
